(19) United States
(12) Patent Application Publication
(10) Pub. No.: US 2004/0181248 A1
(43) Pub. Date: Sep. 16, 2004

(54) Suction round knife

(76) Inventor: Gary D. Josephson, Jacksonville, FL (US)

Correspondence Address: CHARLES E. BAXLEY, ESQ., 90 JOHN STREET, THIRD FLOOR, NEW YORK, NY, 10038, US

(21) Appl. No.: 10/798,122

(22) Filed: Mar. 10, 2004

Related U.S. Application Data
(60) Provisional application No. 60/454,531, filed on Mar. 13, 2003.

(51) Int. Cl. A61B 17/32

(52) U.S. Cl. 606/167

(57) ABSTRACT

A surgical round knife for connecting to a suction source. The knife includes a shaft that is hollow, a round blade on one end of the shaft, and a suction interface on the other end of the shaft. The suction interface fluidly communicates with the suction source so as to provide a suction at the round blade and thereby free up a hand of a surgeon from having to hold a separate suction device and thereby allow the hand of the surgeon to hold an additional instrument. The suction interface includes a bulbous ferrule that is sealing engaged in a plastic suction tube that fluidly communicates with the suction source so as to allow the suction to extend through the shaft. The shaft extends into the round blade approximately one third to one half the round blade so as to provide the suction at the round blade.

### CROSS REFERENCE TO RELATED APPLICATIONS

The instant application is a nonprovisional application of provisional application No. 60/454,531, filed on Mar. 13, 2003, by Gary Josephson, for a JOSEPHSON SUCTION ROUND KNIFE, and from which priority is claimed.

### BACKGROUND OF THE INVENTION

1. Field of the Invention

The present invention relates to a surgical round knife, more particularly, the present invention relates to a surgical round knife for connecting to a suction source.

2. Description of the Prior Art

Presently numerous instruments are required for the successful completion of otologic (ear) surgery. One such instrument is called the round knife. This circular blade is attached to a long shaft, and allows the ear surgeon to make an incision in the ear canal and carefully dissect and lift the ear canal skin off the bony canal wall, down to the tympanic membrane (ear drum) to allow access to the middle ear space. Presently, the surgeon holds the round knife in one hand and an otologic suction in the other while working under the microscope in the ear canal.

Thus, there exists a need for a surgical round knife that frees the hand of the surgeon holding the otologic suction to hold an additional instrument while working under the microscope in the ear canal.

Numerous innovations for surgical instruments have been provided in the prior art. Even though these innovations may be suitable for the specific individual purposes to which they address, they each differ in structure and/or operation and/or purpose from the present invention.

FOR EXAMPLE, U.S. Pat. No. 3,913,584 to Walchle et al. teaches a surgical instrument that constitutes a myringotomy scalpel, aspirator, and otological vent tube inserter including a hand grip member having a trigger hingedly mounted thereto; an aspirator tube projecting outwardly from the hand grip member and having a free, outer end which terminates in a scalpel-defining tip; and a sleeve slidably carried by the aspirator tube and connected to the trigger, wherein pivotal movement of the trigger imparts linear movement to the sleeve along the aspirator tube.

ANOTHER EXAMPLE, U.S. Pat. No. 4,445,517 to Feild teaches a surgical device having aspiration and instrument control. The device is adapted for one-handed simultaneous or alternate dissection and aspiration through a combination of control elements. The device is particularly well-suited for neurosurgery and microsurgery.

STILL ANOTHER EXAMPLE, U.S. Pat. No. to Jeffers et al. teaches a surgical knife having a handle and a blade, the blade having a blunt tip, a generally V-shaped cutting portion extending from the blunt tip to the widest part of the blade and tapered portion extending from the widest part of the blade to the point of attachment of the blade with the handle.

YET ANOTHER EXAMPLE, U.S. Pat. No. 5,709,698 to Adams et al. teaches a shaver blade assembly useable in either an irrigation-only mode, an aspiration-only mode, or an irrigation/aspiration mode. The shaver blade assembly has a stationary elongated outer tube and a movable (e.g. rotatable) elongated inner tube, both inner and outer tubes having hubs attached to their proximal ends for attachment to a handpiece which provides power to move the inner blade relative to the outer blade. The inner and outer tubes are provided with cutting windows at their distal tips and the relative movement between these cutting windows acts to cut tissue during surgical procedures. The outer tube is provided with a fluid inlet port at the proximal end of its tubular surface and a fluid adapter is selectively attachable to the outer tube so as to provide a means for introducing irrigating fluid into the fluid port. The adapter is integrally formed with sealing surfaces which obviate the need for O-rings and the like. A longitudinally extending irrigating channel is provided between the inlet fluid port at one end of the irrigating channel and the outlet fluid port at the distal end of the irrigating channel by which fluid is communicated to the vicinity of the cutting windows when the shaver blade is used in the irrigation-only mode or the irrigation/aspiration mode. The channel may be either the annular clearance space itself or a separate, dedicated groove or channel formed in the mating surfaces of the tubular members. Removing the adapter and the fluid supply enables the shaver to continue being used in an aspiration-only mode.

STILL YET ANOTHER EXAMPLE, U.S. Pat. No. 6,679,897 to Josephson teaches an adenoid curette that includes a shank, a handle, and a cutting head. The shank has a pin, male threads, and a collar. The cutting head is a loop that is continuous and has a groove, a slot, and a neck. The shank is placed in the groove and in the neck, and the pin is positioned in the slot. The collar is threaded onto the male threads, captures the neck during threading, and abuts against the cutting head during threading so as to force the pin further into the slot until the collar can no longer rotate indicating that the pin is fully seated in the slot and thereby causing the cutting head to be captured between the pin and the collar and thereby interchangeably retaining the cutting head on the shank.

It is apparent that numerous innovations for surgical instruments have been provided in the prior art that are adapted to be used. Furthermore, even though these innovations may be suitable for the specific individual purposes to which they address, they would not be suitable for the purposes of the present invention as heretofore described.

### SUMMARY OF THE INVENTION

ACCORDINGLY, AN OBJECT of the present invention is to provide a surgical round knife for connecting to a suction source that avoids the disadvantages of the prior art.

ANOTHER OBJECT of the present invention is to provide a surgical round knife for connecting to a suction source that is simple to use.

BRIEFLY STATED, STILL ANOTHER OBJECT of the present invention is to provide a surgical round knife for connecting to a suction source. The knife includes a shaft that is hollow, a round blade on one end of the shaft, and a suction interface on the other end of the shaft. The suction interface fluidly communicates with the suction source so as to provide a suction at the round blade and thereby free up a hand of a surgeon from having to hold a separate suction device and thereby allow the hand of the surgeon to hold an additional instrument. The suction interface includes a bulbous ferrule that is sealing engaged in a plastic suction tube that fluidly communicates with the suction source so as to allow the suction to extend through the shaft. The shaft extends into the round blade approximately one third to one half the round blade so as to provide the suction at the round blade.

The novel features which are considered characteristic of the present invention are set forth in the appended claims. The invention itself, however, both as to its construction and its method of operation, together with additional objects and advantages thereof, will be best understood from the following description of the specific embodiments when read and understood in connection with the accompanying drawing.

### BRIEF DESCRIPTION OF THE DRAWING

The figures of the drawing are briefly described as follows:

FIG. 1 is a diagrammatic top plan view of the surgical round knife for connecting to a suction source of the present invention; and

FIG. 2 is a diagrammatic side elevational view taken generally in the direction of ARROW 2 in FIG. 1.

FIGS. 3A, 3B, 3C, 3D, 3E, 3F, 3G and 3H depict an otologic surgical procedure in which the present invention is useful.

### LIST OF REFERENCE NUMERALS UTILIZED IN THE DRAWING

10 surgical round knife of present invention for connecting to suction source 12

12 suction source

14 shaft

16 round blade

18 suction interface for fluidly communicating with suction source 12 so as to provide suction at round blade 16 and thereby free up hand (not shown) of surgeon (not shown) from having to hold separate suction device (not shown) and allow hand (not shown) of surgeon (not shown) to hold additional instrument (not shown)

20 proximal end of shaft 14

22 distal end of shaft 14

24 bulbous ferrule of suction interface 18 for being sealing engaged in plastic suction tube 26 that fluidly communicates with suction source 12 so as to allow suction to extend through shaft 14

26 plastic suction tube

### DETAILED DESCRIPTION OF THE INVENTION

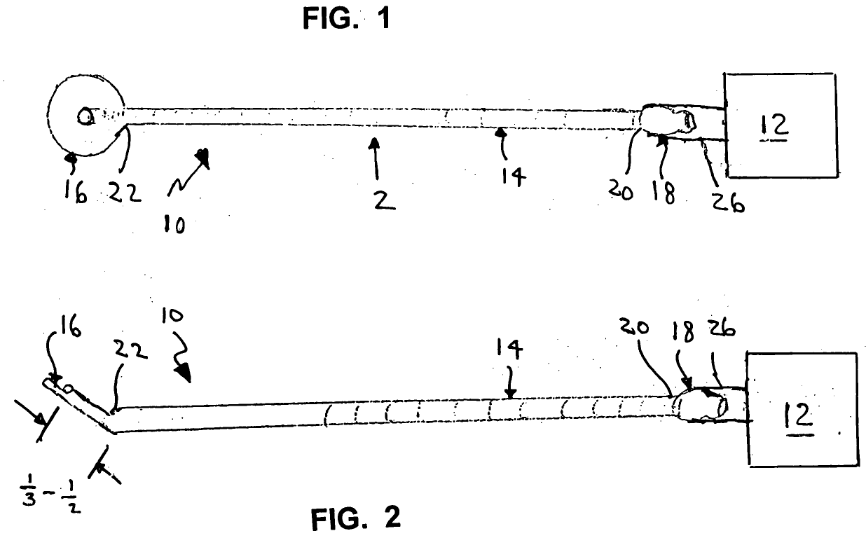

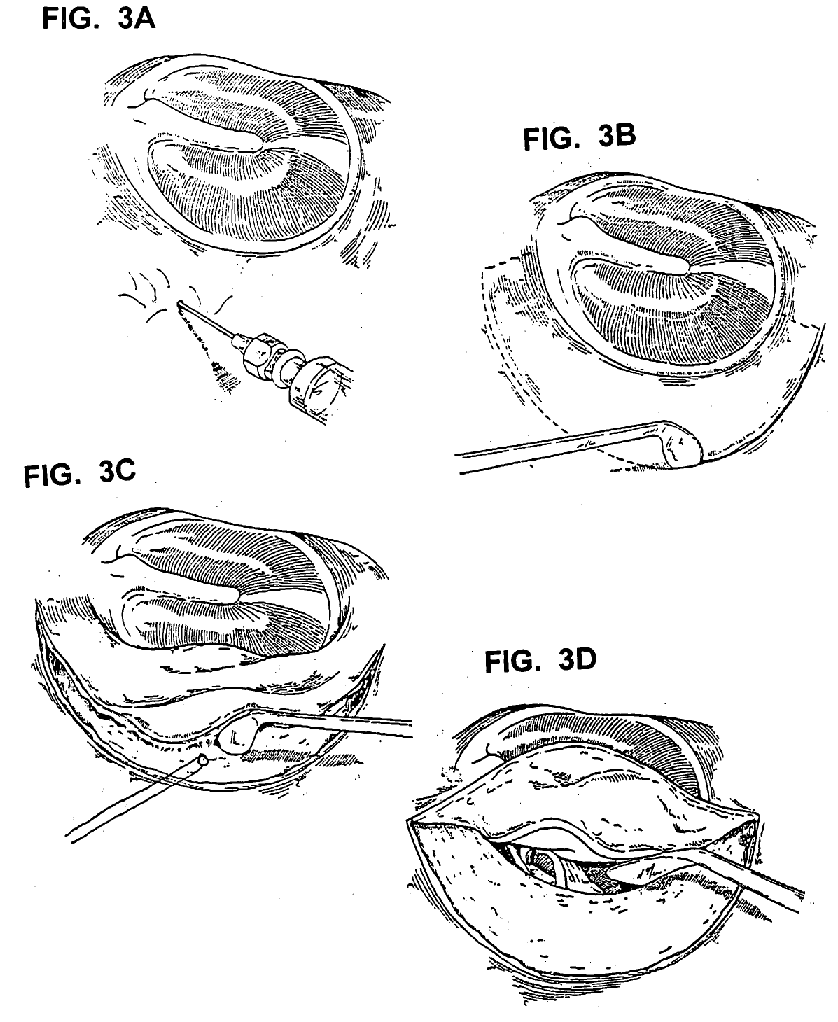

Referring now to the FIGS. 1 and 2, which are, respectively, a diagrammatic top plan view of the surgical round knife for connecting to a suction source of the present invention, and, a diagrammatic side elevational view taken generally in the direction of ARROW 2 in FIG. 1, and in which like numerals indicate like parts, the surgical round knife of the present invention is shown generally at 10 for connecting to a suction source 12.

The surgical round knife 10 comprises a shaft 14, a round blade 16, and a suction interface 18. The round blade 16 is disposed on one end of the shaft 14. The suction interface 18 is disposed on the other end of the shaft 14 and is for fluidly communicating with the suction source 12 so as to provide a suction at the round blade 16 and thereby free up a hand (not shown) of a surgeon (not shown) from having to hold a separate suction device (not shown) and thereby allow the hand (not shown) of the surgeon (not shown) to hold an additional instrument (not shown).

The shaft 14 is slender, elongated, and hollow, and has a proximal end 20 and a distal end 22.

The suction interface 18 is disposed on the proximal end 20 of the shaft 14 and the round blade 16 is disposed on the distal end 22 of the shaft 14.

The suction interface 18 comprises a bulbous ferrule 24. The bulbous ferrule 24 of the suction interface 18 is disposed around the proximal end 20 of the shaft 14 and is for being sealing engaged in a plastic suction tube 26 that fluidly communicates with the suction source 12 so as to allow the suction to extend through the shaft 14.

The bulbous ferrule 24 of the suction interface 18 can be separate from, or be one-piece with, the proximal end 20 of the shaft 14 so long as the bulbous ferrule 24 and the proximal end 20 of the shaft 14 form an integral structure.

The distal end 22 of the shaft 14 extends into the round blade 16 so as to allow the suction to extend through the shaft 14 to the round blade, and terminates approximately one third to one half along the round blade 16.

It will be understood that each of the elements described above, or two or more together, may also find a useful application in other types of constructions differing from the types described above.

Example of Typical Otologic Surgical Procedure In Which the Present Invention is Useful

Otologic surgery requires the skilled surgeon to have a keen sense of vision while working through a microscope, a strong knowledge of the anatomy of the ear and temporal bone, a delicate touch while working around vital structures, and the correct tools to achieve the best results for the patient. As we enter the twenty first century, newer instrumentation is allowing the surgeon to achieve these excellent results in improving hearing, while removing and curing ear disease.

There are two ways that the otologic surgeon gains entrance into the middle and inner ear. One is the trans-canal approach (through the ear canal), and the other is post-auricular (behind the ear). Either of these routes is essential for the surgeon to gain exposure to do his or her work. The transcanal route is more limited, however, either of these techniques require similar instrumentation. The procedures included below are examples of the types of otologic surgery in which the suction round knife is needed.

As in any surgery, one limitation the surgeon experiences is the use of only his or her two hands. In some types of surgery, the surgeon has the fortune of an assistant to help during his or her work allowing additional hands to hold instrumentation. In microscopic otologic surgery, the surgeon does not have the ability to have an assistant because of the small working area. Therefore, the otologist must rely on instrumentation available and only his or her two hands.

In the practice of ear surgery, during most of a procedure, the surgeon finds him or herself using one hand to hold a suction to evacuate blood while the other hand cuts, elevates flaps, and removes or repairs disease, tissue or bone. Hence, the benefit of the new instrument the suction round knife described in this patent application.

Surgeries that can utilize the new suction round knife:

1) Tympanoplasty (repair of eardrum)

2) Ossiculoplasty (repair of ear bones)

3) Mastoidectomy (remove disease (tumor or infection) from mastoid)

4) Cochlear implantation (implant device to provide hearing in deaf patients)

5) Congenital ear surgery (create an ear canal)

6) Inner ear surgeries for dizziness, facial nerve disorders

7) Other similar procedures

Surgical Technique

The technique is described from the point of elevating the skin from the external auditory canal, as the post-auricular and transcanal approach both reach this point at some time during the surgical procedure. The entire procedure is performed looking through a microscope.

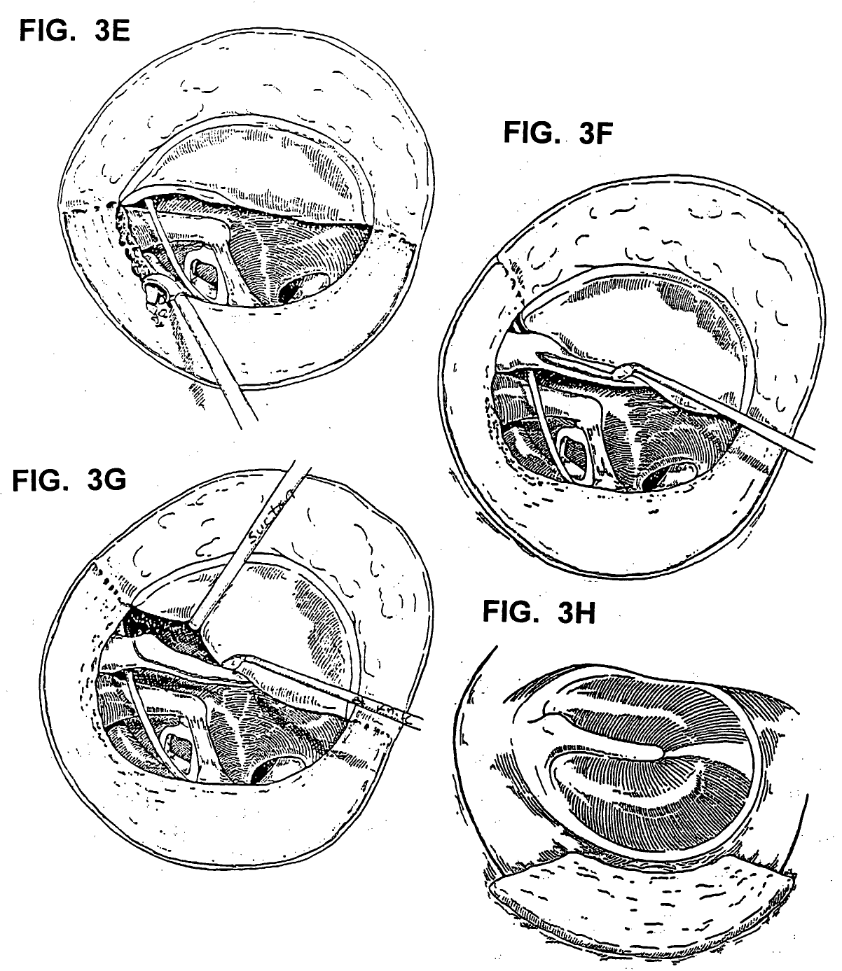

After injection with topical anesthesia into the ear canal (FIG. 3A) and behind the surgeon will now be ready to make the incision and elevate a tympanomeatal flap (ear canal skin and drum). This will allow for exposure to the middle and inner ear structures. The surgeon will use a knife to make two vertical canal incisions. One at 12 O'clock and another at 6 O'clock extending from the ear drum out of the canal about one centimeter. A thir incision will now be made in an horizontal fashion along the posterior canal wall connecting the first two vertical incisions (FIG. 3B). The surgeon uses one hand to hold a suction, and the other hand to hold a tissue elevator (FIG. 3C). In a sequential fashion, the surgeon elevates the tissue off of the bony canal wall while holding the suction tip close to the elevator to evacuate the blood and maintaining visualization. Occasionally, the surgeon will stop to grab another instrument such as a scissor and cut adherent tissue while continuing to elevate or suction depending on how well hemostasis (bleeding) is controlled. Once the skin is elevated off the posterior canal wall down to the ear drum edge, the surgeon delicately elevates the eardrum edge out of its sulcus (FIG. 3D). This allows entrance into the middle ear space. The surgeon proceeds depending on the nature of the problem.

This demonstrates the use of standard instrumentation for the elevation of the tympanometal flap common in most otologic surgery. With the suction round knife, the surgeon has the ability to free one hand so that elevation and suctioning of blood can be performed with a single instrument and hand, allowing the surgeon to use the other hand for additional instrumentation and owk. This allows for a safer, more rapid, and precise elevation of the canal skin in otologic surgery.

Other Uses for the Suction Round Knife

Multiple procedures are mentioned above, however for additional detail, during the entire procedure after the flap is elevated, healthy and diseased tissue is teased off of otologic structures such as the ear bones, floor of the middle ear, mastoid bone, semicircular canals, cochlea, facial nerve, and other structures (FIGS. 3E, 3F 3G and 3H). During these surgical techniques, the surgeon is using a suction, an elevator, and knife by rotating the instruments amongst two hands. Again, this new, useful and non-obvious instrument allows the surgeon to have three instruments in a single hand. The suction round knife has a cutting edge, a round surface for elevation and a suction all in one. This allows the surgeons free hand to use other instrumentation to improve the surgeons ability to perform these operations.

FIGS. 3A-3H are from:

Surgical Pediatric Otolaryngology Editors: Potsic, W P, Cotton R T, Handler S. Dak. Thieme Publishers 1997

Although the invention has been illustrated and described as embodied in a surgical round knife for connecting to a suction source, however, it is not limited to the details shown, since it will be understood that various omissions, modifications, substitutions and changes in the forms and details of the device illustrated and its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute characteristics of the generic or specific aspects of this invention.

The invention claimed is:

1. A surgical round knife for connecting to a suction source, comprising:
a) a shaft;
b) a round blade; and
c) a suction interface;
wherein said round blade is disposed on one end of said shaft;
wherein said suction interface is disposed on the other end of said shaft; and
wherein said suction interface is for fluidly communicating with the suction source so as to provide a suction at said round blade and thereby free up a hand of a surgeon from having to hold a separate suction device and thereby allow the hand of the surgeon to hold an additional instrument.

2. The knife as defined in claim 1, wherein said shaft is slender;
wherein said shaft is elongated; and
wherein said shaft is hollow.

3. The knife as defined in claim 1, wherein said shaft has a proximal end;
wherein said shaft has a distal end;
wherein said suction interface is disposed on said proximal end of said shaft; and
wherein said round blade is disposed on said distal end of said shaft.

4. The knife as defined in claim 1, wherein said suction interface is for being sealing engaged in a plastic suction tube that fluidly communicates with the suction source so as to allow the suction to extend through said shaft.

5. The knife as defined in claim 1; further comprising a plastic suction tube; and wherein said suction interface is sealing engaged in said plastic suction tube that is for fluidly communicating with the suction source so as to allow the suction to extend through said shaft.

6. The knife as defined in claim 1, wherein said shaft has a proximal end;
wherein said suction interface comprises a bulbous ferrule;
wherein said bulbous ferrule of said suction interface is disposed around said proximal end of said shaft; and
wherein said bulbous ferrule of said suction interface is for being sealing engaged in a plastic suction tube that fluidly communicates with the suction source so as to allow the suction to extend through said shaft.

7. The knife as defined in claim 6, wherein said bulbous ferrule of said suction interface is separate from said proximal end of said shaft; and
wherein said bulbous ferrule and said proximal end of said shaft form an integral structure.

8. The knife as defined in claim 6, wherein said bulbous ferrule of said suction interface is one-piece with said proximal end of said shaft; and
wherein said bulbous ferrule and said proximal end of said shaft form an integral structure.

9. The knife as defined in claim 1, wherein said shaft has a distal end; and
wherein said distal end of said shaft extends into said round blade so as to allow the suction to extend through said shaft to said round blade.

10. The knife as defined in claim 1, wherein said shaft has a distal end; and
wherein said distal end of said shaft extends into said round blade approximately one third to one half said round blade.
